(19) United States
(12) Patent Application Publication
(10) Pub. No.: US 2016/0193179 A1
(43) Pub. Date: Jul. 7, 2016

(54) Treatment of Cardiovascular Risk in Diabetic Patients

(71) Applicants: Kjell Steinar SAKARIASSEN, Biella (IT); Alexandra Santana SORENSEN, Moncarapacho (PT); Norbert BENDER, Reinach (CH); EVOLVA SA, Reinach (CH)

(72) Inventors: Kjell Steinar SAKARIASSEN, Biella (IT); Alexandra Santana SORENSEN, Moncarapacho (PT); Norbert BENDER, Reinach (CH)

(21) Appl. No.: 14/653,092

(22) PCT Filed: Dec. 17, 2013
(86) PCT No.: PCT/IB2013/003249

§ 371 Date: Jun. 17, 2015

Related U.S. Application Data
(60) Provisional application No. 61/738,619, filed on Dec. 18, 2012.
(60) Provisional application No. 61/781,953, filed on Mar. 14, 2013.

(51) Int. Cl. A61K 31/357; A61K 45/06

(52) U.S. Cl. CPC A61K 31/357; A61K 45/06

(57) ABSTRACT

Disclosed herein are methods of treating cardiovascular risk in diabetic patients using a pharmaceutically acceptable form of (Z)-6-((2S,4S,5R)-2-(2-chlorophenyl)-4-(2-hydroxyphenyl)-1,3-dioxan-5-yl)hex-4-enoic acid in combination with a pharmaceutically acceptable form of a P2Y12 inhibitor.

### CROSS-REFERENCE TO RELATED APPLICATIONS

This application claims benefit of U.S. Provisional Application Ser. No. 61/781,953, filed Mar. 14, 2013, and U.S. Provisional Application Ser. No. 61/738,619, filed Dec. 18, 2012, the disclosures of both of which are incorporated herein by reference in their entireties.

### BACKGROUND OF THE INVENTION

1. Field of the Invention

The invention relates to a method of treating cardiovascular risk in diabetic patients comprising administering to a diabetic patient a therapeutically effective amount of a pharmaceutically acceptable form of a compound which is (Z)-6-((2S,4S,5R)-2-(2-chlorophenyl)-4-(2-hydroxyphenyl)-1,3-dioxan-5-yl)hex-4-enoic acid in combination with administering to the diabetic patient a therapeutically effective amount of a pharmaceutically acceptable form of a P2Y12 inhibitor.

2. Description of Related Art

According to the World Health Organization (WHO) the total number of people with diabetes was 171 million in 2000, and is projected to rise to 366 million in 2030. Type 2 diabetes is the most common form of diabetes. Diabetic complications are manifestations of systemic disease which affect 80% or more of all diabetic patients during their lifetime. The longer a person has diabetes, the higher the chances of not only developing one of these complications separately but developing a combination of these, which then might themselves become treatment emergencies. Diabetic complications can be classified broadly as microvascular, macrovascular or neuropathic. Microvascular complications include neuropathy (nerve damage), nephropathy (kidney disease) and vision disorders (e.g., retinopathy, glaucoma, cataract and corneal disease). Macrovascular complications include heart disease, stroke and peripheral vascular disease (which can lead to ulcers, gangrene and amputation). Neuropathic complications can include autonomic and peripheral, and include deformation and amputation of the feet or lower legs. Difficulty healing accompanies all of these conditions.

The true prevalence of diabetic complications has been difficult to determine, and these complications are even harder to treat.

Current treatments and prophylaxis of diabetes do not focus on, or target, the complications that accompany diabetes. Atherothrombosis is a significant complication accompanying diabetes disease. Monotherapy treatments and prophylaxis of atherothrombosis such as antiplatelet agents, cyclooxygenase inhibitors and P2Y12 receptor antagonists do nothing to mitigate the effects of prostanoids and of non-enzymatically formed substances such as isoprostanes.

Because a significant number of diabetic patients are at significant risk of developing vascular complications in a variety of organs, including the heart, brain, and kidneys, current therapeutic approaches are urgently needed in managing diabetic disease and diabetic patients.

Thus, there is a continuing need for a therapy or combinations of therapies for the prophylactic, acute treatment and chronic treatment of diabetes and diabetic complications.

### SUMMARY OF THE INVENTION

It is against the above background that the present invention provides certain advantages and advancements over the prior art.

Although the invention as set forth herein is not limited to specific advantages or functionalities, it is noted that in several embodiments the invention provide methods for reducing risk of cardiovascular event in diabetic patients comprising administering to a patient a therapeutically effective amount of a pharmaceutically acceptable form of a compound which is (Z)-6-((2S,4S,5R)-2-(2-chlorophenyl)-4-(2-hydroxyphenyl)-1,3-dioxan-5-yl)hex-4-enoic acid in combination with administering to a patient a pharmaceutically effect amount of a pharmaceutically acceptable form of a P2Y12 inhibitor.

The compound (Z)-6-((2S,4S,5R)-2-(2-chlorophenyl)-4-(2-hydroxyphenyl)-1,3-dioxan-5-yl)hex-4-enoic acid is a dual thromboxane receptor (TP) antagonist and thromboxane synthase (TS) inhibitor.

In further embodiments, the P2Y12 inhibitor is selected from cangrelor, prasugrel, ticagrelor, elinogrel, clopidogrel (Plavix®), or ticlopidine, and combinations thereof, and an amount of a pharmaceutically acceptable form of a compound which is (Z)-6-((2S,4S,5R)-2-(2-chlorophenyl)-4-(2-hydroxyphenyl)-1,3-dioxan-5-yl)hex-4-enoic acid.

In further embodiments, (Z)-6-((2S,4S,5R)-2-(2-chlorophenyl)-4-(2-hydroxyphenyl)-1,3-dioxan-5-yl)hex-4-enoic acid and the P2Y12 inhibitor are administered simultaneously.

In further embodiments, (Z)-6-((2S,4S,5R)-2-(2-chlorophenyl)-4-(2-hydroxyphenyl)-1,3-dioxan-5-yl)hex-4-enoic acid and the P2Y12 inhibitor are administered sequentially.

In further embodiments, the therapeutically effective amount of (Z)-6-((2S,4S,5R)-2-(2-chlorophenyl)-4-(2-hydroxyphenyl)-1,3-dioxan-5-yl)hex-4-enoic acid is a dose from about 0.0001 mg to about 20 mg of (Z)-6-((2S,4S,5R)-2-(2-chlorophenyl)-4-(2-hydroxyphenyl)-1,3-dioxan-5-yl)hex-4-enoic acid per kg of the patient body weight per day.

In further embodiments, the therapeutically effective amount of (Z)-6-((2S,4S,5R)-2-(2-chlorophenyl)-4-(2-hydroxyphenyl)-1,3-dioxan-5-yl)hex-4-enoic acid is a dose of from about 0.001 mg to about 1 mg of (Z)-6-((2S,4S,5R)-2-(2-chlorophenyl)-4-(2-hydroxyphenyl)-1,3-dioxan-5-yl)hex-4-enoic acid per kg of the patient body weight per day.

In some embodiments, the cardiovascular event is myocardial infarction, thrombosis, a thrombotic disorder, stent triggered thrombus formation, stent induced restenosis, stent-triggered hyperplasia, pulmonary hypertension, atherosclerosis, diabetic nephropathy, retinopathy, diabetic retinopathy, peripheral arterial disease, lower limb circulation, pulmonary embolism, thrombus formation, hyperplasia, septic shock, preeclampsia, asthma, rhinitis, allergic rhinitis, tumor angiogenesis or metastasis.

Other embodiments disclose a method for treating vascular inflammation in a diabetic patient comprising administering to the patient a therapeutically effect amount of a pharmaceutically acceptable form of a compound which is (Z)-6-((2S,4S,5R)-2-(2-chlorophenyl)-4-(2-hydroxyphenyl)-1,3-dioxan-5-yl)hex-4-enoic acid in combination with administering to the patient a pharmaceutically effect amount of a pharmaceutically acceptable form of a P2Y12 inhibitor.

Other embodiments disclose a method for reducing vascular inflammation in a diabetic patient comprising administering to the patient a therapeutically effect amount of a pharmaceutically acceptable form of a compound which is (Z)-6-((2S,4S,5R)-2-(2-chlorophenyl)-4-(2-hydroxyphenyl)-1,3-dioxan-5-yl)hex-4-enoic acid in combination with administering to the patient a pharmaceutically effect amount of a pharmaceutically acceptable form of a P2Y12 inhibitor.

Other embodiments disclose a method for reducing platelet reactivity in a diabetic patient comprising administering to the patient a therapeutically effect amount of a pharmaceutically acceptable form of a compound which is (Z)-6-((2S,4S,5R)-2-(2-chlorophenyl)-4-(2-hydroxyphenyl)-1,3-dioxan-5-yl)hex-4-enoic acid in combination with administering to the patient a pharmaceutically effect amount of a pharmaceutically acceptable form of a P2Y12 inhibitor.

Other embodiments disclose a method for improving vascular function in a diabetic patient comprising administering to the patient a therapeutically effect amount of a pharmaceutically acceptable form of a compound which is (Z)-6-((2S,4S,5R)-2-(2-chlorophenyl)-4-(2-hydroxyphenyl)-1,3-dioxan-5-yl)hex-4-enoic acid in combination with administering to the patient a pharmaceutically effect amount of a pharmaceutically acceptable form of a P2Y12 inhibitor.

Other embodiments disclose a method for reducing vascular inflammation and oxidative stress in a diabetic patient comprising administering to the patient a therapeutically effect amount of a pharmaceutically acceptable form of a compound which is (Z)-6-((2S,4S,5R)-2-(2-chlorophenyl)-4-(2-hydroxyphenyl)-1,3-dioxan-5-yl)hex-4-enoic acid in combination with administering to the patient a pharmaceutically effect amount of a pharmaceutically acceptable form of a P2Y12 inhibitor.

These and other features and advantages of the present invention are more fully understood from the following detailed description of the invention taken together with the accompanying drawings and the claims. It is noted that the scope of the claims is defined by the recitations therein and not by the specific discussion of features and advantages set forth in the present description.

The following abbreviations are used in this disclosure:

PD means pharmacodynamic(s)
TBS means tert-butylamine salt
TP means thromboxane receptor
TS means thromboxane synthase
TxA₂ means Thromboxane A2
TxB₂ means Thromboxane B2
CVD means cerebral vascular diseases
TIA means transient ischemic attack
AA means arachidonic acid
PRP means platelet rich plasma
PPP means platelet-poor plasma
MEA means Multiplate Analyzer
LTA means light transmission aggregometry
hs-CRP means C-reactive protein
FPA means fibrin peptide A
ADP means adenosine diphosphate

### BRIEF DESCRIPTION OF THE DRAWINGS

FIGS. 1 and 2A show the effects of Compound 3 on mRNA expression of genes of interest encoding inflammatory or oxidative markers. mRNA expression is evaluated by quantitative RT-PCR in HUVEC after 6 h of TNFα stimulation. Data are means±S.D. (n=3). Statistical analysis: one way ANOVA followed by Bonferroni, Versus CTL NS p>0.05, * p<0.05, ** p<0.01, *** p<0.001; Versus TNFα $ p<0.05, $$ p<0.01, $$$ p<0.001.

FIGS. 2B and 2C show the effect of Compound 3 on VCAM-1 and ICAM-1 protein expression induced by TNFα in HUVEC after 6 h of incubation. B) Total protein extracts are recovered and analysed by western blot. β-actin serves as the loading control. C) Quantification of optical density of each band. Results are expressed as the ratio between the optical density of VCAM-1 or ICAM-1 and the optical density of β-actin.

FIG. 3A shows the effects of Compound 3 and TNFα on 6-keto PGF1α secretion by HUVEC after 6 h of incubation. 6-ketoPGF1α secretion is evaluated using a specific EIA assay. Results are expressed in picograms of 6-ketoPGF1α reported to micrograms of proteins measured by the Folin method on cell lysates. Data are means±S.D. (n=3). Statistical analysis: one way ANOVA followed by Bonferroni, Versus CTL, * p<0.05.

FIG. 3B shows the effects of Compound 3 and TNFα on PTX3 secretion in HUVEC after 6 h of incubation. PTX3 secretion is evaluated using a specific ELISA. Results are expressed in picograms of PTX3 reported to micrograms of proteins measured by the Folin method on cell lysates. Data are means±S.D. (n=3). Statistical analysis: one way ANOVA followed by Bonferroni, Versus CTL, NS p>0.05.

FIGS. 3C and 3D show the effect of Compound 3 on PARP cleavage induced by TNFα in HUVEC after 6 h of incubation. C) Total protein extracts are recovered and analysed by western blot. β-actin serves as the loading control. D) Quantification of optical density of each band and expression of the ratio between the optical density of PARP cleavage and the total of PARP.

FIGS. 4 and 5A show the effects of Compound 3 and TNFα on thrombin generation in HUVEC after 6 h of incubation. Thrombin generation is evaluated using CAT assay. Results express active thrombin concentration (nM) along the time (min) when coagulation is induced by: A) 5 pM TF (tissue factor) and 4 μM PL (phospholipids), B) 1 pM TF and 4 μM PL, C) 4 μM PL. D) and E) Separated graphs for Lag time and active thrombin concentration when coagulation is induced by 4 μM PL. Data are expressed as means±1 S.D. (n=3).

FIG. 5B shows the effect of the TP receptor agonist U46619 on the proliferation of human coronary artery smooth muscle cells, assessed by WST-1 reagent. Experiments are performed either in the presence of FCS (2%), or in the presence of FCS (5%), FGF (2 ng/ml), EGF (0.5 ng/ml), and insulin (5 ng/ml). Results are expressed as mean±SE for n=3-7 observations at each concentration. All points are significantly different (P<0.05) when comparing the two groups.

FIG. 6A shows the effect of the dual TP receptor antagonist and thromboxane synthase inhibitor Compound 3 on the proliferation of human coronary artery smooth muscle cells induced by U46619, assessed by WST-1 reagent. All experiments are performed in the presence of FCS (5%), FGF (2 ng/ml), EGF (0.5 ng/ml), and insulin (5 μg/ml). Results (mean±SE) are expressed as fold change compared with vehicle treated cells (in the absence of U46619), n=7. *P<0.05 vs. Control at each concentration.

FIG. 6B shows a comparison of platelet aggregation in citrate-anticoagulated PRP (LTA) of patients on aspirin treatment with and without Compound 3.

FIG. 7A shows a comparison of platelet aggregation in citrate-anticoagulated PRP (LTA) of patients on clopidogrel treatment with and without Compound 3.

FIG. 7B shows a comparison of platelet aggregation in citrate-anticoagulated PRP (LTA) patients treated with Compounds 3 and either aspirin or clopidogrel.

### DETAILED DESCRIPTION OF THE INVENTION

All publications, patents and patent applications cited herein are hereby expressly incorporated by reference for all purposes.

Before describing the present invention in detail, a number of terms are defined. As used herein, the singular forms “a”, “an”, and “the” include plural referents unless the context clearly dictates otherwise.

It is noted that terms like “preferably”, “commonly”, and “typically” are not utilized herein to limit the scope of the claimed invention or to imply that certain features are critical, essential, or even important to the structure or function of the claimed invention. Rather, these terms are merely intended to highlight alternative or additional features that may or may not be utilized in a particular embodiment of the present invention.

“Optional” and “optionally” mean that the subsequently described event or circumstance mayor may not occur, and that the description includes instances where the event or circumstance occurs and instances in which it does not. For example, optional pharmaceutical excipients indicates that a formulation so described may or may not include pharmaceutical excipients other than those specifically stated to be present, and that the formulation so described includes instances in which the optional excipients are present and instances in which they are not.

“Treating” and “treatment” refer to any treatment of a disease or the complications arising from a disease in a mammal, particularly a human, and include:
(i) preventing the disease and/or complications from occurring in a subject which may be predisposed to the disease but has not yet been diagnosed as having it;
(ii) inhibiting the disease or complications, i.e., arresting its development, progression, or prevalence; or
(iii) relieving the disease or complications, i.e., causing regression of the disease or complications.

In certain aspects, “treating” refers to any treatment of a disease or the complications arising from a disease in a mammal, particularly a human, and include:
(ii) inhibiting the disease or complications, i.e., arresting its development, progression, or prevalence; or
(iii) relieving the disease or complications, i.e., causing regression of the disease or complications.

As used herein, the phrase “therapeutically effective amount” refers to the amount of active compound or pharmaceutical agent that elicits the biological or medicinal response that is being sought in a tissue, system, animal, individual or human by a researcher, veterinarian, medical doctor or other clinician.

Modified release formulations as used herein refer to pharmaceutical formulations designed to release the drug or pharmaceutical in a manner that is different than an immediate release formulation. Thus, a modified release formulation may be a controlled release, sustained release, extended release, or delayed release formulation. Particular modified release formulations suitable for use in the invention are extended release formulations that release a drug or compound in the body over an extended period of time.

Compound 1 is a racemic mixture of (Z)-6-((2S,4S,5R)-2-(2-chlorophenyl)-4-(2-hydroxyphenyl)-1,3-dioxan-5-yl)hex-4-enoic acid and (Z)-6-((2R,4R,5S)-2-(2-chlorophenyl)-4-(2-hydroxyphenyl)-1,3-dioxan-5-yl)hex-4-enoic acid as described in WO 2008/089461. The synthesis of Compound 1 is described in Example 1 of WO 2008/089461; see Scheme 2, structure 2-14.

Compound 2 is (Z)-6-((2S,4S,5R)-2-(2-chlorophenyl)-4-(2-hydroxyphenyl)-1,3-dioxan-5-yl)hex-4-enoic acid, a single enantiomer of Compound 1. The separation of Compound 2 from its enantiomer is described in Example 30 of WO 2008/089461.

Compound 3 is tert-butylammonium (Z)-6-((2S,4S,5R)-2-(2-chlorophenyl)-4-(2-hydroxyphenyl)-1,3-dioxan-5-yl)hex-4-enoate (the tert-butylamine salt (TBS) of Compound 2) as described in WO 2008/089461. Compound 3 is a dual thromboxane receptor (TP) antagonist and thromboxane synthase (TS) inhibitor. The preparation of Compound 3 is described in Example 36 of WO 2008/089461.

Compound 2 is a highly active TP antagonist and a thromboxane synthase (TS) inhibitor, however, its use as a pharmaceutical agent has been limited by poor solubility and bioavailability. Compound 3 is a highly soluble and crystalline salt of Compound 2, and is a highly effective vehicle for the delivery of Compound 2.

Compound 3 has been shown to antagonize TP receptor activation in vivo at concentrations ranging from 0.125 to 2.5 mg/kg. See Angiolillo, D. J., et al. Journal of Thrombosis and Thrombolysis 2012 34(3):297-9. DOI:10.1007/s11239-012-0795-6 and Sorensen, A. S. et al. Eur J Clin Pharmacol. 2013 69(3):459-65. DOI:10.1007/s00228-012-1348-9. The disclosures of both articles are incorporated herein by reference in their entirety

Disclosed herein, are methods for treating cardiovascular events such as, for example, thrombotic disorders, vascular inflammation, vascular oxidative stress, arterial thrombotic events, diabetic nephropathy, diabetic neuropathy, complications of peripheral arterial diseases, hyperglycemia, hypoglycemia, increased ketones, cardiovascular diseases including myocardial infarction, acute coronary syndrome, angina, percutaneous coronary intervention, cerebrovascular diseases (CVD) such as stroke and transient ischemic attacks (TIAs), carotid stenosis, diabetic macro- and micro-angiopathy including peripheral vascular ischemia, peripheral artery disease, arterial thrombosis, intermittent claudication, diabetic ulcers and foot ulcers, diabetes related skin disorders, diabetic retinopathy including non-proliferative retinopathy and proliferative retinopathy, diabetic renal diseases including micro- and macro proteinuria, nephrotic syndrome, nephritic syndrome impaired renal function such as reduced GFR, increased creatinine, and reduced clearance, end stage renal failure, Raynaud's disease and/or syndrome, and diabetic microcirculation, including foot ulcers and disorders leading to peripheral amputations.

The disclosed invention provides methods for reducing risk of cardiovascular event in diabetic patients comprising administering to a patient a therapeutically effective amount of a pharmaceutically acceptable form of a compound which is (Z)-6-((2S,4S,5R)-2-(2-chlorophenyl)-4-(2-hydroxyphenyl)-1,3-dioxan-5-yl)hex-4-enoic acid in combination with administering to a patient a pharmaceutically effect amount of a pharmaceutically acceptable form of a P2Y12 inhibitor.

In further embodiments, the P2Y12 inhibitor is selected from cangrelor, prasugrel, ticagrelor, elinogrel, clopidogrel (Plavix®), or ticlopidine, and combinations thereof, and an amount of a pharmaceutically acceptable form of a compound which is (Z)-6-((2S,4S,5R)-2-(2-chlorophenyl)-4-(2-hydroxyphenyl)-1,3-dioxan-5-yl)hex-4-enoic acid.

In further embodiments, (Z)-6-((2S,4S,5R)-2-(2-chlorophenyl)-4-(2-hydroxyphenyl)-1,3-dioxan-5-yl)hex-4-enoic acid and the P2Y12 inhibitor are administered simultaneously.

In further embodiments, (Z)-6-((2S,4S,5R)-2-(2-chlorophenyl)-4-(2-hydroxyphenyl)-1,3-dioxan-5-yl)hex-4-enoic acid and the P2Y12 inhibitor are administered sequentially.

In further embodiments, the therapeutically y effective amount of (Z)-6-((2S,4S,5R)-2-(2-chlorophenyl)-4-(2-hydroxyphenyl)-1,3-dioxan-5-yl)hex-4-enoic acid is a dose from about 0.0001 mg to about 20 mg of (Z)-6-((2S,4S,5R)-2-(2-chlorophenyl)-4-(2-hydroxyphenyl)-1,3-dioxan-5-yl)hex-4-enoic acid per kg of the patient body weight per day.

In further embodiments, the therapeutically y effective amount of (Z)-6-((2S,4S,5R)-2-(2-chlorophenyl)-4-(2-hydroxyphenyl)-1,3-dioxan-5-yl)hex-4-enoic acid is a dose of from about 0.001 mg to about 1 mg of (Z)-6-((2S,4S,5R)-2-(2-chlorophenyl)-4-(2-hydroxyphenyl)-1,3-dioxan-5-yl)hex-4-enoic acid per kg of the patient body weight per day.

In some embodiments, the cardiovascular event is myocardial infarction, thrombosis, a thrombotic disorder, stent triggered thrombus formation, stent induced restenosis, stent-triggered hyperplasia, pulmonary hypertension, atherosclerosis, diabetic nephropathy, retinopathy, diabetic retinopathy, peripheral arterial disease, lower limb circulation, pulmonary embolism, thrombus formation, hyperplasia, septic shock, preeclampsia, asthma, rhinitis, allergic rhinitis, tumor angiogenesis or metastasis.

Other embodiments disclose a method for treating vascular inflammation in a diabetic patient comprising administering to the patient a therapeutically effect amount of a pharmaceutically acceptable form of a compound which is (Z)-6-((2S,4S,5R)-2-(2-chlorophenyl)-4-(2-hydroxyphenyl)-1,3-dioxan-5-yl)hex-4-enoic acid in combination with administering to the patient a pharmaceutically effect amount of a pharmaceutically acceptable form of a P2Y12 inhibitor.

Other embodiments disclose a method for reducing vascular inflammation in a diabetic patient comprising administering to the patient a therapeutically effect amount of a pharmaceutically acceptable form of a compound which is (Z)-6-((2S,4S,5R)-2-(2-chlorophenyl)-4-(2-hydroxyphenyl)-1,3-dioxan-5-yl)hex-4-enoic acid in combination with administering to the patient a pharmaceutically effect amount of a pharmaceutically acceptable form of a P2Y12 inhibitor.

Other embodiments disclose a method for reducing platelet reactivity in a diabetic patient comprising administering to the patient a therapeutically effect amount of a pharmaceutically acceptable form of a compound which is (Z)-6-((2S,4S,5R)-2-(2-chlorophenyl)-4-(2-hydroxyphenyl)-1,3-dioxan-5-yl)hex-4-enoic acid in combination with administering to the patient a pharmaceutically effect amount of a pharmaceutically acceptable form of a P2Y12 inhibitor.

Other embodiments disclose a method for improving vascular function in a diabetic patient comprising administering to the patient a therapeutically effect amount of a pharmaceutically acceptable form of a compound which is (Z)-6-((2S,4S,5R)-2-(2-chlorophenyl)-4-(2-hydroxyphenyl)-1,3-dioxan-5-yl)hex-4-enoic acid in combination with administering to the patient a pharmaceutically effect amount of a pharmaceutically acceptable form of a P2Y12 inhibitor.

Other embodiments disclose a method for reducing vascular oxidative stress in a diabetic patient comprising administering to the patient a therapeutically effect amount of a pharmaceutically acceptable form of a compound which is (Z)-6-((2S,4S,5R)-2-(2-chlorophenyl)-4-(2-hydroxyphenyl)-1,3-dioxan-5-yl)hex-4-enoic acid in combination with administering to the patient a pharmaceutically effect amount of a pharmaceutically acceptable form of a P2Y12 inhibitor.

In the disclosed methods, the pharmaceutically acceptable form of (Z)-6-((2S,4S,5R)-2-(2-chlorophenyl)-4-(2-hydroxyphenyl)-1,3-dioxan-5-yl)hex-4-enoic acid is an amorphous form, a crystalline form, a salt, a polymorph, a co-crystal, a solvate, or a prodrug.

In certain aspects, the pharmaceutically acceptable form useful in the invention comprises Compound 2 (as the acid).

In certain aspects of the disclosed methods of the invention, the pharmaceutically acceptable form may be a crystalline or amorphous salt of Compound 2 selected from, e.g., tert-butylamine salts, sodium salts, potassium salts, arginine salts, lysine salts, calcium salts, zinc salts, magnesium salts, choline salts, ethylenediamine salts, ethanolamine salts, ammonium salts, hydroxyethylpyrrolidine salts, dimethylaminoethanol salts, hydroxyethylmorpholine salts, N-ethylglucamine salts, N-methylglucamine salts, tromethamine salts, imidazole salts, or wherein the compound is a di-salt on both the carboxylic acid and the phenol moieties, such as, for example a di-sodium and a di-potassium salt.

Yet further in the disclosed methods, the co-crystal is selected from hydrates of salt(s), ethanol, 1-butanol, isopropanol and dioxane solvates of Compound 3, co-crystals of Compound 3 with glycerol, methyl tert-butyl ether (MTBE) solvates of Compound 3, and the tert-butylamine salt of a racemic mixture of (Z)-6-((2S,4S,5R)-2-(2-chlorophenyl)-4-(2-methoxyphenyl)-1,3-dioxan-5-yl)hex-4-enoic acid and (Z)-6-((2R,4R,5S)-2-(2-chlorophenyl)-4-(2-methoxyphenyl)-1,3-dioxan-5-yl)hex-4-enoic acid.

In certain aspects of the disclosed methods of the invention, the pharmaceutically acceptable form may be a prodrug. “Prodrug” refers to a derivative of an active compound (drug) that undergoes a transformation under the conditions of use, such as within the body, to release an active drug. Prodrugs are frequently, but not necessarily, pharmacologically inactive until converted into the active drug. Prodrugs are typically obtained by masking a functional group in the drug believed to be in part required for activity with a “progroup”. Progroups are typically attached to the functional group of the drug via bonds that are cleavable under specified conditions of use. The cleavage of the progroup may proceed spontaneously, such as by way of a hydrolysis reaction, or it may be catalyzed or induced by another agent, such as by an enzyme, by light, by acid, or by a change of or exposure to a physical or environmental parameter, such as a change of temperature, or combination thereof. The agent may be endogenous to the conditions of use, such as an enzyme present in the cells to which the prodrug is administered or the acidic conditions of the stomach, or it may be supplied exogenously.

In addition to or in place of Compound 2 or Compound 3, the compound is a dual thromboxane receptor (TP) antagonist and thromboxane synthase (TS) inhibitor selected from compounds of Formula I:

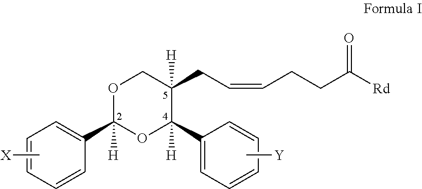

wherein

X is selected from the group consisting of fluoro, chloro, bromo, trifluoromethyl, substituted phenyl, cyano, methoxy, nitro, hydroxyl and —H; and Y is selected from the group consisting of fluoro, chloro, bromo, trifluoromethyl, substituted phenyl, cyano, methoxy, nitro, hydroxyl, —C(O)-saccharide and —H; and Rd is —NH—C₁₋₆-alkyl (branched or linear, preferably linear), —O—C₁₋₆-alkyl (branched or linear, preferably linear), a saccharide or —OH.

In addition to or in place of Compound 2 or Compound 3, the dual thromboxane receptor (TP) antagonist and thromboxane synthase (TS) inhibitor may further be selected from compounds of Formula II:

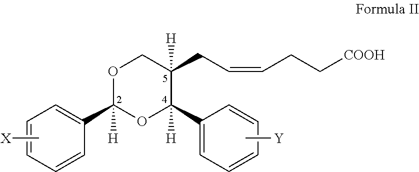

wherein

X is selected from the group consisting of fluoro, chloro, bromo, trifluoromethyl, substituted phenyl, cyano, methoxy, nitro, hydroxyl and —H; and Y is selected from the group consisting of fluoro, chloro, bromo, trifluoromethyl, substituted phenyl, cyano, methoxy, nitro, hydroxyl, —C(O)-saccharide and —H; and Rd is —NH—C₁₋₆-alkyl (branched or linear, preferably linear), —O—C₁₋₆-alkyl (branched or linear, preferably linear), a saccharide or —OH.

In addition to or in place of Compound 2 or Compound 3, the dual thromboxane receptor (TP) antagonist and thromboxane synthase (TS) inhibitor may further be selected from compounds of Formula III:

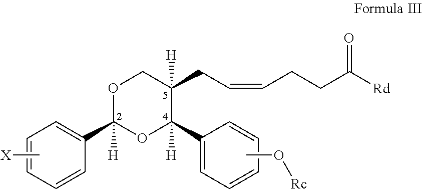

wherein

X is selected from fluoro, chloro, bromo, trifluoromethyl, optionally substituted phenyl, cyano, methoxy and nitro; Rc is C₁₋₆-alkyl (branched or linear, preferably linear), —C(O)—Ci₋₆-alkyl (branched or linear, preferably linear), —CH(O), a saccharide or —H; and Rd is —NH—C₁₋₆-alkyl (branched or linear, preferably linear), —O—C₁₋₆-alkyl (branched or linear, preferably linear), glycosyl or —OH.

In addition to or in place of Compound 2 or Compound 3, the dual thromboxane receptor (TP) antagonist and thromboxane synthase (TS) inhibitor may further be selected from compounds of Formula IV:

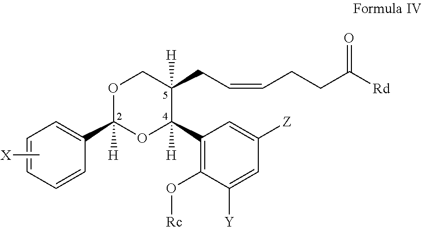

wherein
X, Z and Y are as specified herein above for compounds of Formula I, preferably X is halogen, more preferably X is chloro, wherein X may be in the ortho, meta and/or para position, preferably the ortho position, and preferably Z and Y are both —H; Rc is C₁₋₆-alkyl (branched or linear, preferably linear), —C(O)—C₁₋₆-alkyl (branched or linear, preferably linear), —CH(O), a saccharide (preferably a mono- or disaccharide, more preferably, a monosaccharide, even more preferably glycosyl) or —H, preferably Rc is methoxy, —C(O)—CH₃ or —H; and Rd is —NH—C₁₋₆-alkyl (branched or linear, preferably linear), —O—C₁₋₆-alkyl (branched or linear, preferably linear), a saccharide (preferably a mono- or disaccharide, more preferably, a monosaccharide, even more preferably glycosyl) or —OH, preferably Rd is —OH, glycosyl or —O—CH₃, more preferably Rd is —H or —O—CH₃.

In addition to or in place of Compound 2 or Compound 3, the dual thromboxane receptor (TP) antagonist and thromboxane synthase (TS) inhibitor may further be selected from compounds of Formula V:

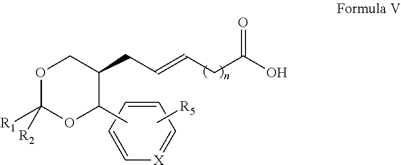

### Wherein

R₁ can be hydrogen, halogen, cyano, hydroxyl, or alkyl; R₂ can be hydrogen, alkyl, alkenyl, aryl, heteroaryl, or a C₃-30 cyclic or heterocyclic ring optionally substituted with one or more substituent; X can be CH, or N; n can be 0, 1, 2, 3, 4, or 5; and R₅ can be H, OH, alkoxy, alkyl, or halogen. The substituent can be H, —R^{a}, halo, —O⁻, ═O, —OR^{b}, —SR^{b}, —S⁻, ═S, —NR^{c}R^{c}, ═NR^{b}, ═N—OR^{b}, trihalomethyl, —CF₃, —CN, —OCN, —SCN, —NO, —NO₂, —N₂, —N₃, —S(O)₂R^{b}, —S(O)₂O⁻, —(CH₂)₀₋₄S(O)₂OR^{b}, —OS(O)₂R^{b}, —OS(O)₂O⁻, —OS(O)₂OR^{b}, —P(O) (OR^{b})(O⁻), —P(O)(O⁻)₂, —P(O)(OR^{b})(OR^{b}), —C(O)R^{b}, —C(S)R^{b}, —C(NR^{b})R^{b}, —C(O)O⁻, —C(O)OR^{b}, —C(S)OR^{b}, —C(O)NR^{c}R^{c}, —C(NR^{b})NR^{c}R^{c}, —OC(O)R^{b}, —OC(S)R^{b}, —OC(O)O⁻, —OC(O)OR^{b}, —OC(S) OR^{b}, —NR^{b}C(O)R^{b}, —NR^{b}C(S)R^{b}, —NR^{b}C(O)O⁻, —NR^{b}C(O)OR^{b}, —NR^{b}C(S)OR^{b}, —NR^{b}C(O)NR^{c}R^{c}, —NR^{b}C(NR^{b})R^{b} and —NR^{b}C(NR^{b})NR^{c}R^{c}, where R^{a} is selected from the group consisting of alkyl, cycloalkyl, heteroalkyl, cycloheteroalkyl, aryl, arylalkyl, heteroaryl and heteroarylalkyl; each R^{b} is independently hydrogen or R^{a}; and each R^{c} is independently R^{b} or alternatively, the two R^{c} groups are taken together with the nitrogen atom to which they are bonded form a 5-, 6- or 7-membered cycloheteroalkyl which may optionally include from 1 to 4 of the same or different additional heteroatoms selected from the group consisting of O, N and S. The C₃₋₃₀ cyclic or heterocyclic ring can be unsubstituted, singly substituted or multiply substituted acenaphthene, benzothiophene, chromanone, indole, julolidine, naphthalene, quinoline, and the like.

The compounds described herein may include functional groups that can be masked with progroups to create prodrugs. Such prodrugs are usually, but need not be, pharmacologically inactive until converted into their active drug form. In the prodrugs of the invention, any available functional moiety may be masked with a progroup to yield a prodrug. Progroups that are cleavable under the desired conditions of use are known in the art.

In the disclosed methods, the dual thromboxane receptor (TP) antagonist and thromboxane synthase (TS) inhibitor may be a prodrug of one or the other, or a mixture, of the enantiomers of Formula VI:

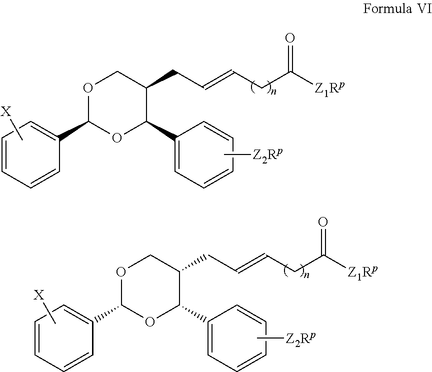

wherein

X can be hydrogen, halogen, cyano, nitro, hydroxyl, haloalkyl, alkyl, or O—R where R is a lower alkyl group; n can be 0, 1, 2, 3, 4, or 5; Z₁ and Z₂ can be independently selected to be O, N, or S, and R^{P} is independently selected from H, lower alkyl, or a progroup. The prodrugs can thus be compounds where both Z₁ and Z₂ can be O, and at least one R^{P} is a progroup such as lower alkyl, ester, amide, and the like. The progroup R^{P} metabolizes in vivo to yield the active diaryl 1,3-dioxane moiety containing drug.

Compounds containing functional groups, such as phenolic, carboxylic, thiolic groups, and the like, can be derivatized with progroups for the synthesis of prodrugs. A wide variety of progroups suitable for masking functional groups in active compounds to yield prodrugs are well-known in the art. For example, a hydroxyl functional group may be masked as a sulfonate, ester or carbonate progroup, which may be hydrolyzed, typically in vivo to provide the hydroxyl group. An amino functional group may be masked as an amide, imine, phosphinyl, phosphonyl, phosphoryl or sulfonyl progroup, which may be hydrolyzed in vivo to provide the amino group. A carboxyl group may be masked as an ester (including silyl esters and thioesters), amide or hydrazide progroup, which may be hydrolyzed in vivo to provide the carboxyl group. Other specific examples of suitable progroups are apparent to those of skill in the art.

For example, when a compound of the present invention contains a carboxylic acid functional group, a prodrug can comprise an ester formed by the replacement of the hydrogen atom of the acid group with a group including, but not limited to, groups such as for example (C₁-C₈)alkyl, (C₂-C₁₂)alkanoyloxymethyl, 1-(alkanoyloxy)ethyl having from 4 to 9 carbon atoms, 1-methyl-1-(alkanoyloxy)-ethyl having from 5 to 10 carbon atoms, alkoxycarbonyloxymethyl having from 3 to 6 carbon atoms, 1-(alkoxycarbonyloxy)ethyl having from 4 to 7 carbon atoms, 1-methyl-1-(alkoxycarbonyloxy)ethyl having from 5 to 8 carbon atoms, N-(alkoxycarbonyl)aminomethyl having from 3 to 9 carbon atoms, 1-(N (alkoxycarbonyl)amino)ethyl having from 4 to 10 carbon atoms, 3-phthalidyl, 4-crotonolactonyl, gamma-butyrolacton-4-yl, di-N,N—(C₁-C₂)alkylamino(C₂-C₃)alkyl, carbamoyl-(C₁-C₂)alkyl, N,N-di(C₁-C₂)alkylcarbamoyl-(C₁-C₂)alkyl and piperidino-, pyrrolidino- or morpholino(C₂-C₃)alkyl. The prodrug may also be a compound wherein an —COOH group has reacted with a saccharide to form an ester, the saccharide preferably being a mono- or disaccharide, more preferably, a monosaccharide, even more preferably glucose. In particular, the phenol group can be converted to a phosphate ester or an alkyl ester, or derivatized using polyethylene glycol (PEG), alkyloxycarbonykloxymethyl (AOCOM), or as a sterically hindered alkoxycarbonyloxymethyl, as illustrated below.

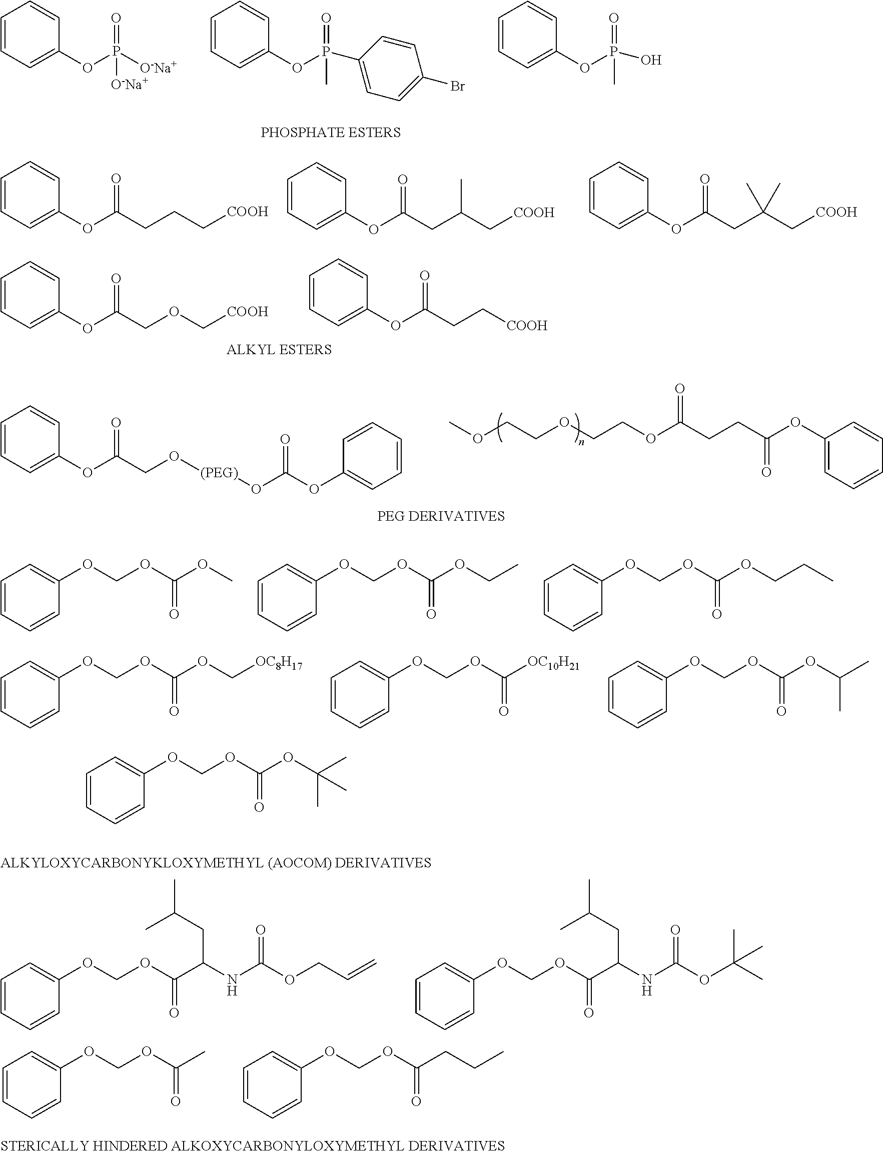

Progroups can be selected to provide a prodrug with the intended water solubility, mode of administration and/or intended mechanism or site of metabolism to the active 2,4-diaryl-1,3-dioxane compound. For example, the progroup can be lipophilic or hydrophilic, where the lipophilic groups can be used to decrease water solubility and hydrophilic groups can be used to increase water solubility. In this way, prodrugs specifically tailored for selected modes of administration can be obtained. The progroup can also be designed to impart the prodrug with other properties, such as, for example, improved passive intestinal absorption, improved transport-mediated intestinal absorption, protection against fast metabolism (slow-release prodrugs), tissue-selective delivery, passive enrichment in target tissues, targeting-specific transporters, etc.

Representative prodrugs useful of the methods of the invention include:

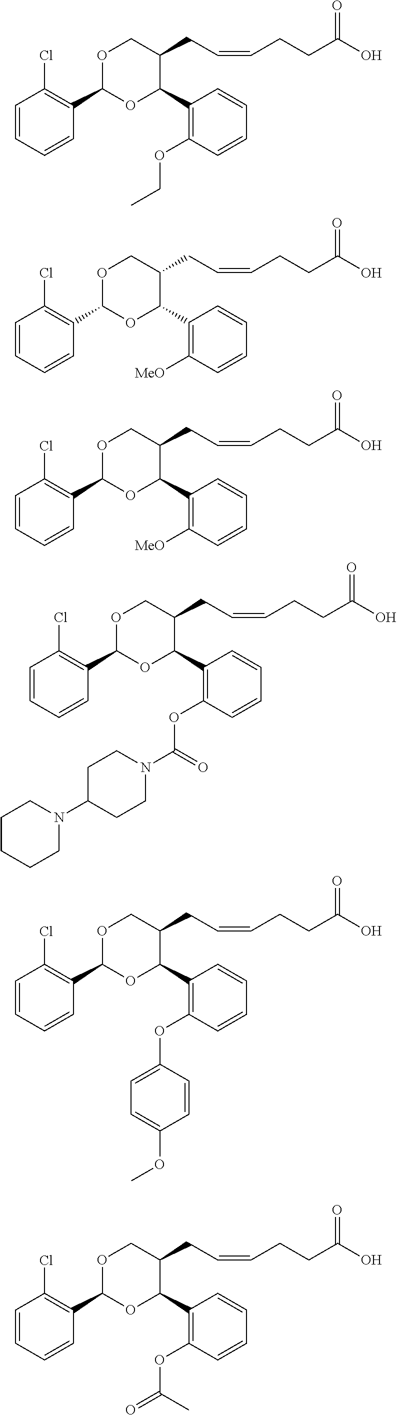 [SMILES: CCOc1ccccc1[C@H]1O[C@@H](c2ccccc2Cl)OC[C@H]1C/C=C\CCC(=O)O ; COc1ccccc1[C@@H]1O[C@H](c2ccccc2Cl)OC[C@@H]1C/C=C\CCC(=O)O ; COc1ccccc1[C@H]1O[C@@H](c2ccccc2Cl)OC[C@H]1C/C=C\CCC(=O)O ; O=C(O)CC/C=C\C[C@@H]1CO[C@H](c2ccccc2Cl)O[C@@H]1c1ccccc1OC(=O)N1CCC(N2CCCCC2)CC1 ; COc1ccc(Oc2ccccc2[C@H]2O[C@@H](c3ccccc3Cl)OC[C@H]2C/C=C\CCC(=O)O)cc1 ; CC(=O)Oc1ccccc1[C@H]1O[C@@H](c2ccccc2Cl)OC[C@H]1C/C=C\CCC(=O)O]

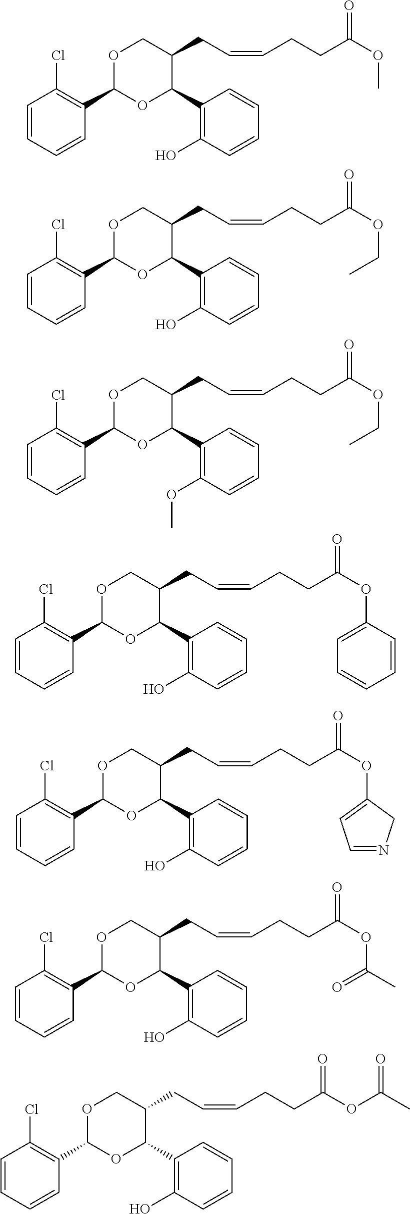

In the disclosed methods, the prodrug is the racemic mixture of (Z)-6-((2S,4S,5R)-2-(2-chlorophenyl)-4-(2-methoxyphenyl)-1,3-dioxan-5-yl)hex-4-enoic acid and (Z)-6-((2R,4R,5S)-2-(2-chlorophenyl)-4-(2-methoxyphenyl)-1,3-dioxan-5-yl)hex-4-enoic acid, (Z)-6-((2S,4S,5R)-2-(2-chlorophenyl)-4-(2-methoxyphenyl)-1,3-dioxan-5-yl)hex-4-enoic acid, (Z)-6-((2S,4S,5R)-4-(2-acetoxyphenyl)-2-(2-chlorophenyl)-1,3-dioxan-5-yl)hex-4-enoic acid, (Z)-methyl 6-((2S,4S,5R)-2-(2-chlorophenyl)-4-(2-hydroxyphenyl)-1,3-dioxan-5-yl)hex-4-enoate, an alkyl, aryl or heteroaryl ester, amide or sulphonamide derivative of the (Z)-6-((2S,4S,5R)-2-(2-chlorophenyl)-4-(2-hydroxyphenyl)-1,3-dioxan-5-yl)hex-4-enoic acid, or an alkyl, aryl and heteroaryl phenolic ether of (Z)-6-((2S,4S,5R)-2-(2-chlorophenyl)-4-(2-hydroxyphenyl)-1,3-dioxan-5-yl)hex-4-enoic acid, or a pharmaceutically acceptable salt thereof. It should be recognized that these inventions also provides embodiments containing varying combinations or all of these expressly-recited features.

The Examples that follow are illustrative of specific embodiments of the invention, and various uses thereof. They set forth for explanatory purposes only, and are not to be taken as limiting the invention.

### EXAMPLES

### Example 1

### Effects of Compound 3 on TNFα-Induced Inflammation in Human Umbilical Vein Endothelial Cells (HUVEC)

### PTX3 ELISA and 6-Keto PGF1α EIA

HUVEC are seeded at 25,000 cells/well on gelatin-coated 24-well plate (Corning) in EGM. After 48 h, cell medium is replaced by EBM containing or not containing Compound 3 at different concentrations. After 1 h of preincubation, 1 ng/ml TNFα is added or is not added to the wells. 6 h after adding TNFα, medium of each well is collected and cell lysis is performed in NaOH 0.5 N for 30 min. PTX3 secretion in the incubation medium is assayed using specific ELISAs (#DPTX30, Quantikine, R&D Systems, USA) according to the procedure provided by the supplier. 6-ketoPGF1α levels in cell culture media are determined using a competitive assay (#515211, Cayman, USA). Results are expressed in picograms of PTX3 or 6-ketoPGF1α reported to micrograms of proteins measured by the Folin method (Folin-Ciocalteu's phenol reagent, Merck, Germany) in cell lysates.

### Western Blot Analysis

HUVEC are seeded at 187,500 cells/flask in gelatin-coated T75 in EGM. After 4 days, cell medium is replaced by EBM containing or not containing Compound 3 at different concentrations. After 1 h of preincubation, 1 ng/ml TNFα is added or is not added into the T75 flasks. 6 h after, cell media are collected and centrifuged at 500 g for 5 min to pellet and pool floating cells with adherent cells. Adherent cells are washed twice with cold PBS and are collected in 1.5 ml of PBS. Harvested cells are then centrifuged at 500 g for 5 min at 4° C. Cells are lysed in 50 μl of SDS lysis buffer (10 mM Tris, pH 7.5; 0.1 mM EDTA; 0.1 mM EGTA, 0.5% SDS; 0.1 mM β-mercaptoethanol, protease inhibitor cocktail (Roche, Germany); phosphatase inhibitor buffer (25 mM Na₃VO₄, 250 mM PNPP, 250 mM β-glycerophosphate, 125 mM NaF)). Protein concentration is measured using Pierce method (Pierce 660 nm protein assay, Thermoscientific, USA) with the Ionic Detergent Compatibility Reagent (Thermoscientific, USA). Equal amounts of protein are separated on 10% SDS-polyacrylamide gel electrophoresis and transferred to PVDF membrane (Immobilon-FL, Millipore Corporation, USA). Membranes are blocked in the LI-COR blocking buffer (Biosciences, USA) for 1 h at room temperature with gentle agitation. Primary antibodies (anti-PARP1 (BD Pharmingen, #551025, 1:1000), anti-VCAM1 (Santa Cruz, #sc-1504, 1:500), anti-ICAM1 (Santa Cruz, #sc-7891, 1:500) and anti-β-actin (Sigma, #A5441, 1:20000)) are diluted in LI-COR blocking buffer supplemented with 0.1% Tween-20 and are incubated overnight at 4° C. Membranes are then washed 4 times, 5 min each, in PBS containing 0.1% Tween-20 and incubated with fluorescent secondary antibodies 1 h at RT, in the darkness, diluted 1:7500 in LI-COR blocking buffer with 0.1% Tween-20. Antibody binding was analysed with the scanner Odyssey Image System (Li-COR, Biosciences, USA) and Odyssey V3.0 software. Optical density of each band is also determined by using the software.

### Real Time RT-PCR

HUVEC are seeded at 67,500 cells/flask in gelatin-coated T25 in EGM. After 4 days, cell medium is replaced by EBM containing or not containing Compound 3 at different concentrations. After 1 h of preincubation, 1 ng/ml TNFα is added or is not into the T25 flasks. 6 h after, cell medium is discarded and cells are washed twice with PBS. Total RNA extraction is performed by using QIAGEN RNeasy mini Kit according to the protocol provided by the manufacturer (QIAGEN). RNA concentration for each sample is determined by Nanodrop Spectrophotometer ND-100 (ISOGEN, Life Science). Reverse transcription was performed using Transcriptor First Strand cDNA Synthesis Kit Roche (Roche Applied Science). Transcript levels are determined by quantitative reverse transcription polymerase chain reaction (qRT-PCR). cDNA obtained by reverse transcription of total RNA are diluted 100 times and 5 μl are mixed with 12.5 μl of Fast start Universal SYBRGreen Master (Roche), 2.5 μl MilliQ water, 2.5 μl of diluted primer forward and 2.5 μl of diluted primer reverse to have an optimal and final concentration of 300 nM or 900 nM. PCRs are carried out in a real-time PCR cycler (Applied Biosystems 7900HT Fast Real-time PCR System): a hot start at 95° C. for 5 min is followed by 40 cycles at 95° C. for 15 s and 65° C. for 1 min. Samples are compared using the relative cycle threshold (Ct) method. To normalize the load of cDNA for each sample, RPL13 is used as the endogenous standard. Primer sequences are listed below.

List of primers used in real time RT-PCR.

| | Forward | Reverse |
|---|---|---|
| COX2 | ATTAGCCTGAATGT | ACCCACAGTGCTT |
| | GCCATAAGACT | GACACAGAAT |
| ICAM-1 | GGAGCTTCGTGTCC | ACATTGGAGTCTG |
| | TGTATGG | CTGGGAATT |
| IL-6 | TGGCTGCAGGACAT | CCATGCTACATTT |
| | GACAA | GCCGAAGA |
| IL-8 | TTGATACTCCCAGT | TGACTGTGGAGTT |
| | CTTGTCATTGC | TTGGCTGTTT |
| MCP-1 | CATTGTGGCCAAGG | AGTGAGTGTTCAA |
| | AGATCTG | GTCTTCGGAGTT |
| PTX3 | CAATGGACTCCATC | GATGAAGAGCTTG |
| | CCACTGA | TCCCATTCC |
| RPL13 | GCCTACAAGAAAGT | TGAGCTGTTTCTT |
| | TTGCCTATCTG | CTTCCGGTAGT |
| VCAM-1 | TTGGCTCACAATTA | GCAGGTATTATTA |
| | AGAAGTTTAACAC | AGGAGGATGCAA |

Calibrated Automated Thrombogram (CAT)

HUVEC are seeded at 25,000 cells/well on gelatin-coated 24-well plate (Corning) in EGM. After 48 h, cell medium is replaced by M199 (not EBM, because of the presence of anti-coagulant particles in EBM) containing or not containing Compound 3 at different concentrations. After 1 h of preincubation, 1 ng/ml TNFα is added or not in the wells. 6 h after adding TNFα, medium of each well is collected.

For the thrombin activity measurements, 80 μL of NPP (Normal Pooled Plasma), and 20 μL of sample are mixed in a 96-well microtiter plate (Thermo Immulon 2HB, Thermo Labsystems, The Netherlands) and are incubated for 5 min at 37° C. Three inducers of coagulation are used in the assay to study either extrinsic pathway, intrinsic pathway or both simultaneously on thrombin generation. The use of 4 μM PL (phospholipids) induces the intrinsic pathway, the combination of 4 μM PL and 5 pM TF (tissue factor) induces the extrinsic pathway while the combination of 4 μM PL and 1 pM TF induces both pathways. Directly after adding inducer into samples, plasma clotting is triggered by the addition of 20 μL of fluorogenic substrate/calcium chloride buffered solution at 37° C. A calibration curve is also performed simultaneously using 80 μL of NPP, 10 μL of PBS, 20 μL of thrombin Calibrator and 20 μL of substrate/calcium chloride-buffered solution at 37° C. The thrombin substrate hydrolysis into a fluorophore is monitored during 60 minutes with a microplate fluorometer Fluoroskan Ascent FL (Thermo Labsystems, The Netherlands) with a 390/460 nm filter set using the Thrombinoscope software (v 3.0, Thrombinoscope BV). Lag time (time for thrombin generation, in minutes) and Cₘₐₓ (maximal concentration of generated thrombin, in nM) parameters are measured.

Effect of Compound 3 on COX-2, IL-6, IL-8, ICAM-1, VCAM-1, PTX3, MCP-1 mRNA Expression Induced by TNFα

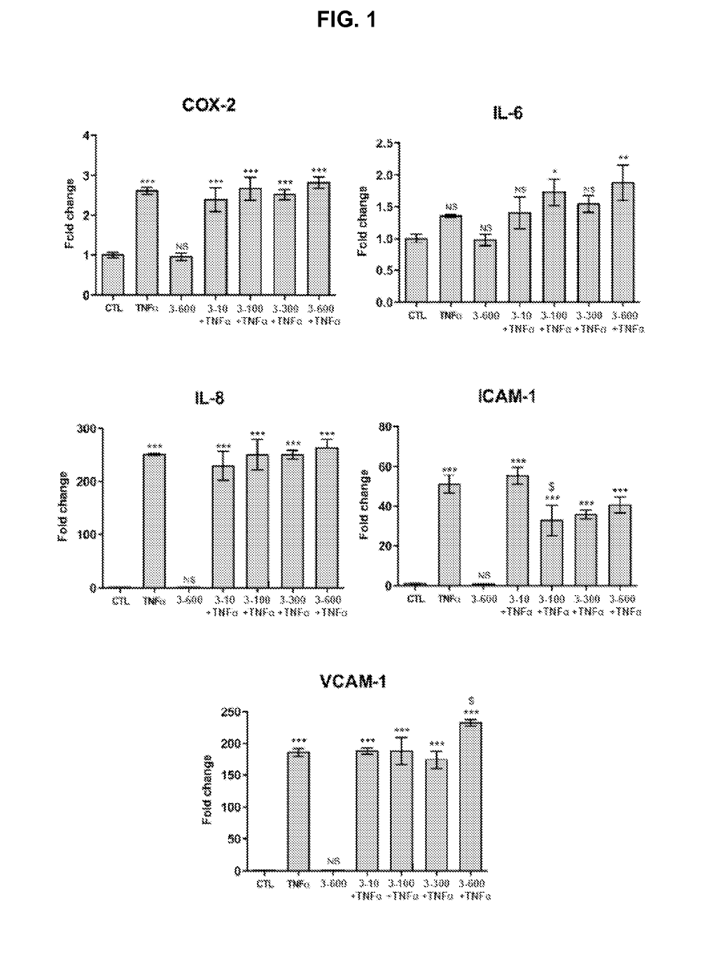

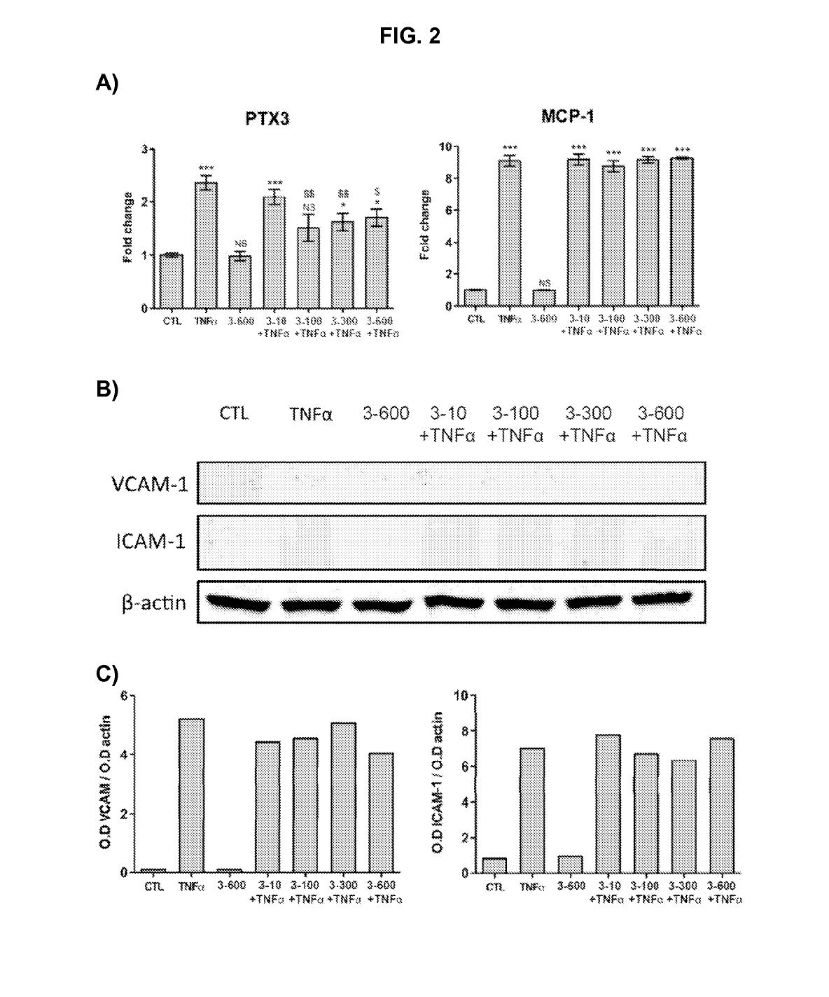

A statistical increase in mRNA expression of all genes of interest is observed upon TNFα stimulation, except for IL-6 for which the increase is not significant. It is also important to note that Compound 3 at the highest concentration has no effect on the mRNA expression of all these genes. Only two genes, ICAM-1 and PTX3, show interesting results with a significant decrease in their mRNA expression by Compound 3 from a concentration of 100 nM (10 nM has no effect). This effect is not increased at higher concentrations (300 nM and 600 nM) (FIGS. 1 and 2A).

### Effect of Compound 3 on VCAM-1 and ICAM-1 Protein Expression Induced by TNFα

Becasue of the observed decrease in ICAM-1 mRNA expression induced by TNFα the ICAM-1 protein level is studied by western blot analysis. The same is done for VCAM-1 in case there is no modification at mRNA level but one at protein level. Optical density of each band is quantified to normalise VCAM-1 and ICAM-1 expression by β-actin expression. Results show that ICAM-1 and VCAM-1 expression increased when cells were stimulated with TNFα for 6 h, but no effect of Compound 3 could be detected (FIGS. 2B and 2C).

### Effect of Compound 3 and TNFα on 6-Keto PGF1α Secretion

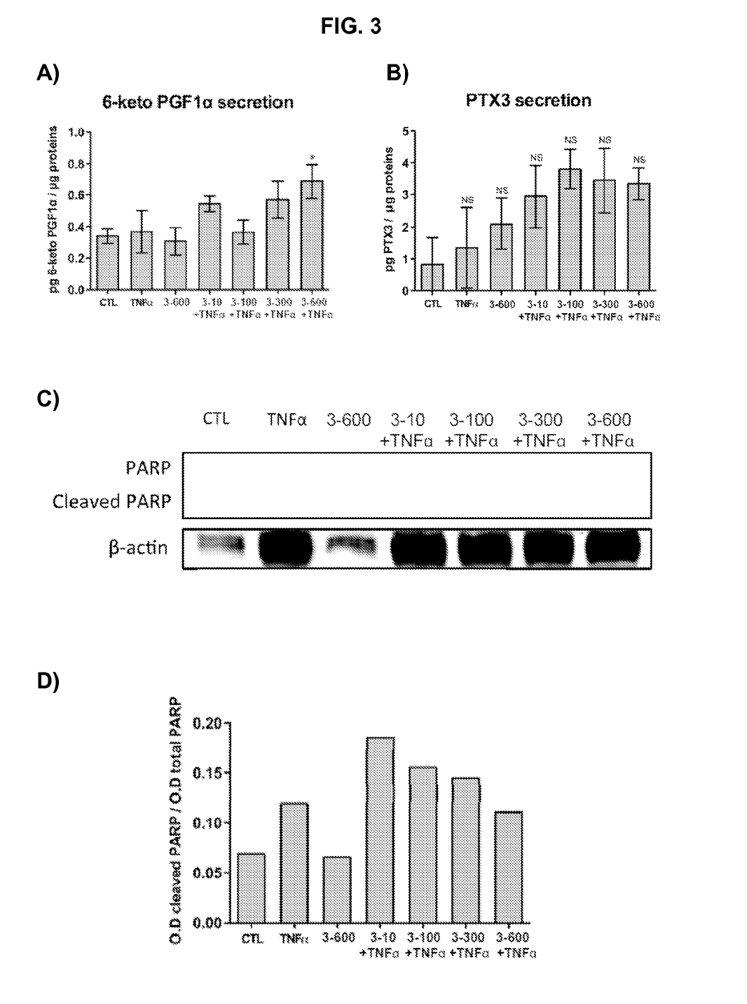

Prostacyclin (PIG₂, Prostaglandin I₂) is formed from arachidonic acid by the vascular endothelium and is a local potent vasodilator and inhibitor of platelet aggregation. PIG₂ is non-enzymatically hydrated to 6-keto PGF1α in endothelial cells. In regard to the interesting role on coagulation, 6-keto PGF1α secretion by HUVEC incubated with Compound 3 and stimulated by TNFα, using a specific EIA is studied. Results show that Compound 3 and TNFα do not increase 6-keto PGF1α secretion, independently of each other. However, secretion seems to be increased when cells are simultaneously incubated with Compound 3 and TNFα, with a significant increase at 600 nM (3-600+TNFα) (FIG. 3A).

### Effect of Compound 3 on PTX3 Secretion

Because of the observed decrease in TNFα-induced PTX3 mRNA expression in the presence of Compound 3 incubation, PTX3 secretion was assayed using a specific ELISA. In contrast to mRNA expression results, no increase in PTX3 secretion is detected in media from cells stimulated by TNFα during 6 h. However, these results are difficult to interpret because the levels of secreted PTX3 are very low, just above blank samples. 6 h of incubation is probably not enough time to accumulate a measurable amount of secreted PTX3, and to reflect variations in mRNA expression. This experiment should be performed again after 24 h of incubation (FIG. 3B).

### Effect of Compound 3 and TNFα on the Apoptosis Level Evaluated by the Cleavage of PARP-1

PARP-1 is a protein that is cleaved by active caspases in cells undergoing apoptosis. PARP cleavage is assessed by western blotting. Results show that TNFα slightly increased PARP cleavage after 6 h (WB scan and quantification), indicating that a small fraction of cells undergo apoptosis upon TNFα stimulation. Compound 3 does not decrease this cleavage induced by TNFα, meaning that Compound 3 does not protect cells from apoptosis induced by TNFα. Further, Compound 3 does not induce apoptosis by itself (FIGS. 3C and 3D).

### Effect of Compound 3 and TNFα on Thrombin Generation

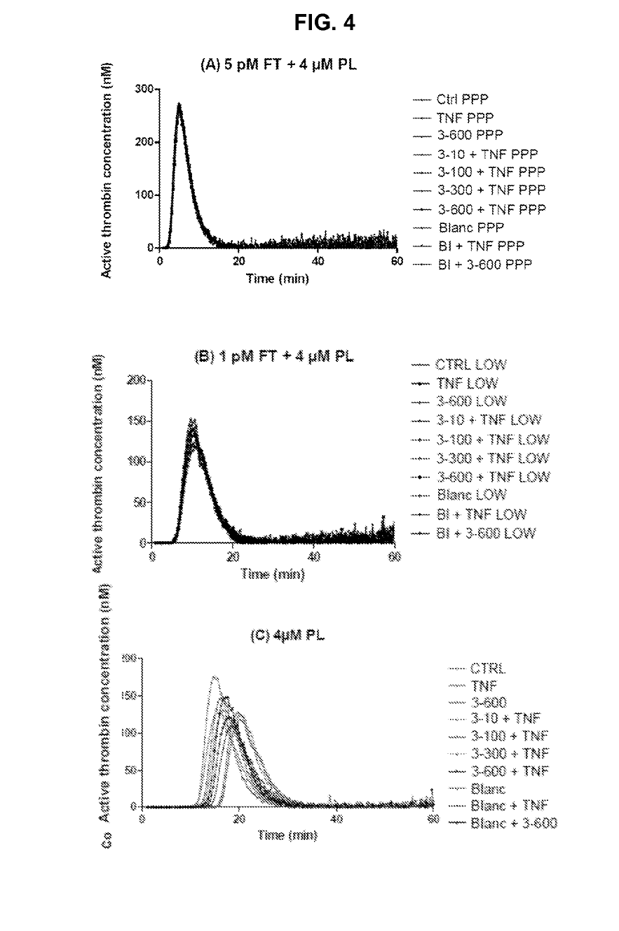

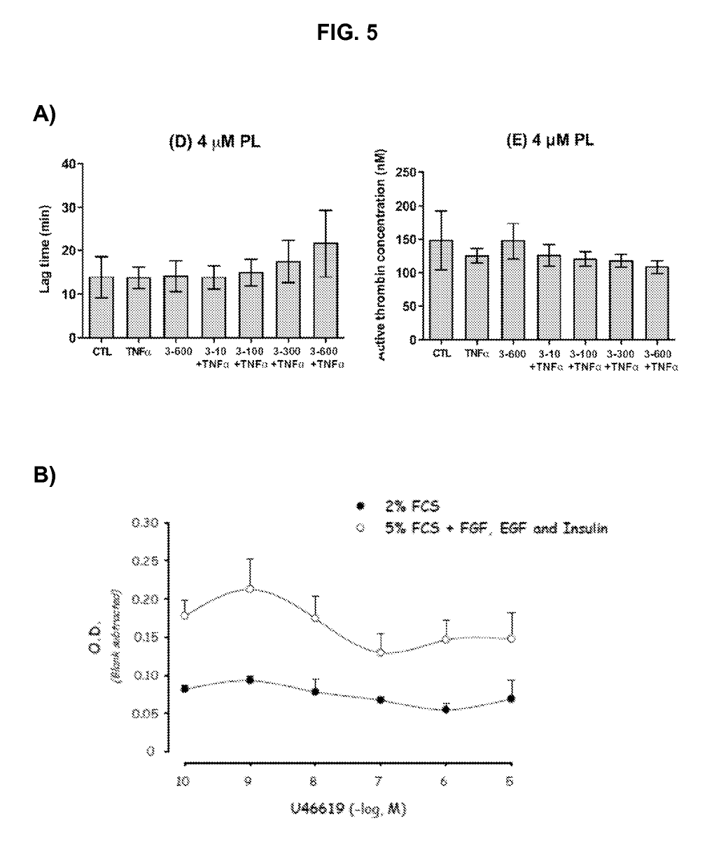

The Calibrated Automated Thrombogram (CAT) is accurate to evaluate the procoagulant potential of a solution that is to say the basal procoagulant activity of particles potentially released by cells in different conditions. Conditioned media are collected after 6 h from HUVEC incubated with Compound 3+/−TNFα in M199 medium. Three inducers of coagulation are used in this assay to study either extrinsic pathway, intrinsic pathway or both simultaneously on thrombin generation. The use of 4 μM PL (phospholipids) induces the intrinsic pathway, the combination of 4 μM PL and 5 pM TF (tissue factor) induces the extrinsic pathway while the combination of 4 μM PL and 1 pM TF induces both pathways. Lag time (time for thrombin generation, in minutes) and Cₘₐₓ (maximal concentration of generated thrombin, in nM) parameters are measured. Results show that Compound 3 and TNFα had no effect on coagulation (extrinsic and intrinsic pathways) (FIGS. 4 and 5A).

### Example 2

### Effect of Compound 3 on Human Coronary Artery Smooth Muscle Cell Proliferation

Human coronary artery SMC is cultured in SmGM2 kit medium. At 80% confluence, the SMCs are trypsinized and resuspended in either DMEM supplemented with 2% FCS (“low serum media”), or in SmGM2 kit medium containing 5% FCS, 2 ng/ml FGF, 0.5 ng/ml EGF, 5 μg/ml insulin (“growth factor media”). Subsequently, 5000 cells in 200 μL are added to each well in a 96-well plate and left to adhere overnight. Either vehicle or Compound 3 (50 nM, 500 nM, 5 μM) is added to the wells 1 h before U 46619 at different concentrations (0.1 nM to 10 μM) and incubated for another 48 h. All experiments are repeated 3-5 times for each experimental condition.

Cell proliferation is evaluated using WST-1 reagent according to the manufacturer's instructions. In brief, after replacement of phenol red containing media by 200 μL transparent serum-supplemented DMEM, 10 μL of diluted WST-1 reagent is added to each well at the end of the 48 h incubation period. After 1 h incubation at 37° C., the absorbance of the formazan dye formed is measured at 440 nm using a microplate reader.

All results are expressed as mean±SE. Statistically significant differences are determined by a one-way analysis of variances, followed by Holm-Sidak post hoc test, for multiple comparisons, using Sigma Stat software. A P value of less than 0.05 is considered significant.

In the presence of growth factor media, the thromboxane mimetic U46619 induces a bell-shaped concentration-dependent increase in cell number as assessed by WST-1 (FIG. 5B). The maximal response is observed at U46619-concentration of 1 nM, and at concentrations of 0.1 μM and higher, the absorbance does not significantly differ between U46619- and vehicle-treated cells (FIG. 5B). In contrast to those findings, U46619 did not significantly alter SMC proliferation when experiments are performed in low serum media (FIG. 5B).

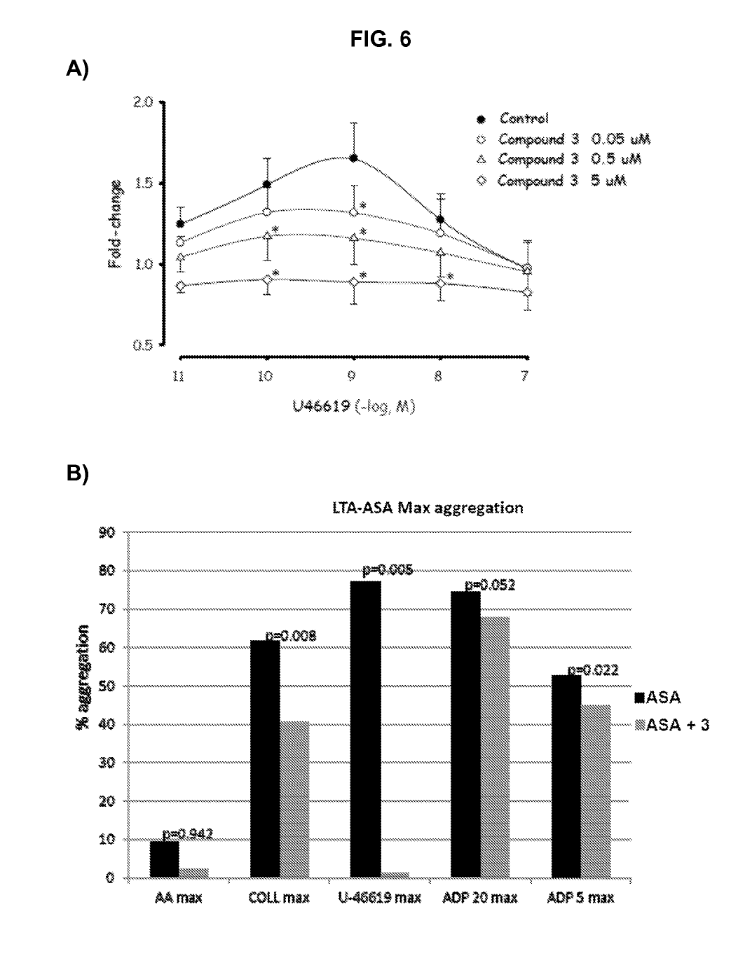

In the presence of growth factor media, the dual TP receptor antagonist and thromboxane synthase inhibitor Compound 3 significantly and concentration-dependently inhibits the SMC proliferation induced by U46619 (FIG. 6A).

### Example 3

### In-Vitro Inhibition of Platelet Aggregation with Compound 3 in Blood of Type-2 Diabetics on Clopidogrel

Patient population: Ten patients affected by type-2 diabetes mellitus and CAD (coronary artery disease), on chronic daily treatment with 81 mg aspirin (enteric coated acetylsalicylic acid, Cardioaspirin, Bayer, Germany) are switched to clopidogrel (75 mg/day) monotherapy for 7-10 days prior to study. Pharmacodynamic assessments are performed while on aspirin (81 mg) monotherapy and clopidogrel (75 mg) monotherapy.

### Blood Sampling and Platelet Aggregation—Light Transmission Aggregometry

Blood sampling with 19-gauge needle or larger without the use of tourniquet. The first two ml of blood sampled was discarded. The venous blood samples are drawn into tri-sodium citrate filled tubes (BD Vacutainer 0.109 M. Ref. 363048) for the LTA. All tri-sodium citrate used to anticoagulate a blood sample remains in the plasma phase since it was not taken up by platelets, erythrocytes and leukocytes. Citrate plasma concentration and the resulting free plasma Ca2+ needed for platelet aggregation is directly determined by the individual haematocrit. Therefore, the plasma citrate concentration and the resulting free plasma Ca2+ concentration has to be adjusted by a volume of 108 mM tri-sodium citrate concentration to a final plasma concentration of 20 mM according to the following formula:

Vc=V(9.2−0.18% Hct)/88, where

Vc=volume of 109 mmol/l tri-sodium citrate added to blood sample anticoagulated with 1/10 volume 109 mmol/l tri-sodium citrate
V=volume of anticoagulated blood sample
% Hct=hematocrit

Platelet-rich plasma (PRP) is prepared by centrifugation at 120×g for 10 min at room temperature. Autologous platelet-poor plasma (PPP) is prepared by centrifugation at 2300×g for 15 min at room temperature. The PRP platelet count is adjusted to 250.000/ul by the autologous PPP. Stability for the platelet aggregation test is 2 hours at room temperature. Platelet aggregation is performed using LTA [2-5]. Blood is collected in citrated tubes. Citrate concentrations are adjusted according to the individual haematocrit levels according to prior LTA studies using Evolva compound. LTA is assessed using PRP by the turbidimetric method in a 2-channel aggregometer (Chrono-Log 490 Model, Chrono-Log Corp., Havertown) according to the instructions of the manufacturer. Light transmission is adjusted to 0% with the PRP and to 100% for the PPP for each measurement. Curves are recorded for 6 minutes. Platelet aggregation is determined as the maximal percent change in light transmittance from baseline using PPP as a reference as well as late platelet aggregation at 6 minutes. Platelet agonists include U-46619 (7 uM), arachidonic acid (1 mM), collagen (3 ug/ml) and ADP (20 μM and 5 μM). Pharmacodynamic testing is performed following 10 min in vitro incubation at 37° C. of PRP with 100 nM Compound 3 and without 100 nM Compound 3 (placebo).

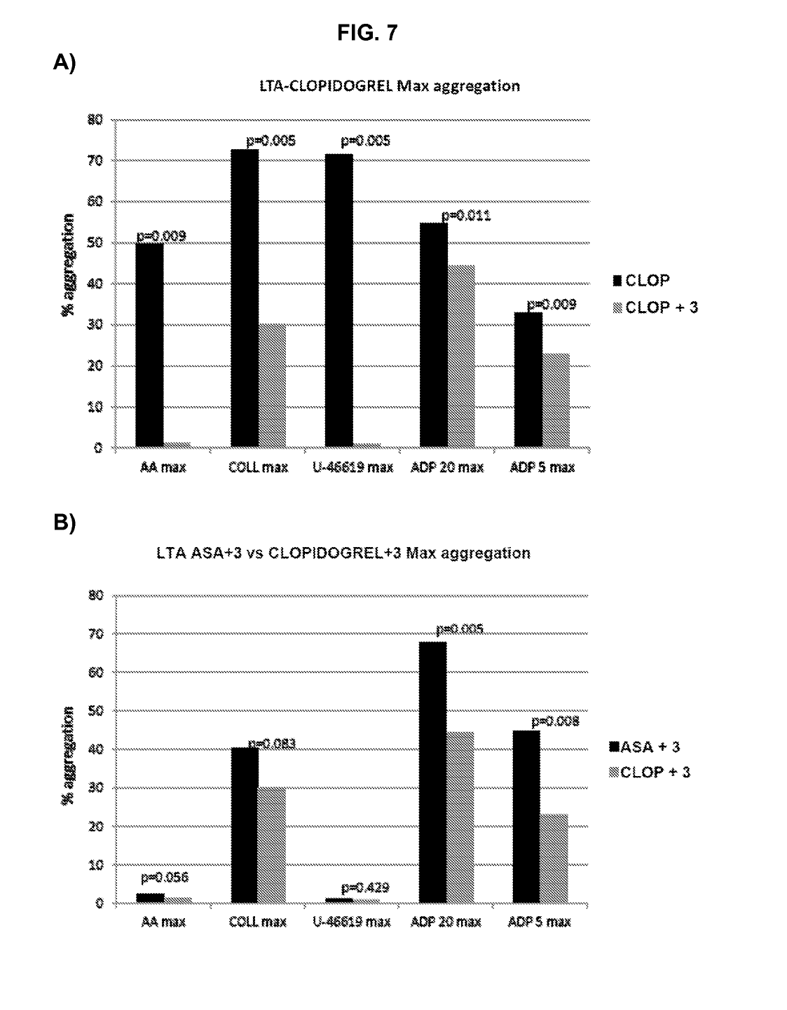

Total of 10 LTA analysis per patient at each time point; total 2 time points=20 assessments. Table 1 and FIG. 6B compare aspirin vs. aspirin+Compound 3, Table 2 and FIG. 7A compare clopidogrel vs. clopidigrel+Comppound 3, and Table 3 and FIG. 7B compare aspirin+Compound 3 vs. clopidogrel+Comound 3.

TABLE 1
Platelet aggregation in citrate-anticoagulated PRP (LTA) on aspirin
treatment with and without Compound 3.

| | ASA (mean ± SD) | ASA + 3 (mean ± SD) | P |
|---|---|---|---|
| AA max | 9.4 ± 22.7 | 2.3 ± 1.2 | 0.942 |
| AA late | 8.5 ± 23 | 1.7 ± 1.6 | 0.797 |
| COLL max | 61.4 ± 17.6 | 40.4 ± 15.5 | 0.008 |
| COLL late | 59.6 ± 17.2 | 39.1 ± 15.5 | 0.005 |
| U-46619 max | 77 ± 5.2 | 1.2 ± 0.7 | 0.005 |
| U-46619 late | 74.9 ± 4.6 | 0 ± 0 | 0.005 |
| ADP 20 max | 74.3 ± 6 | 67.6 ± 8.7 | 0.052 |
| ADP 20 late | 72 ± 6 | 59 ± 20.2 | 0.041 |
| ADP 5 max | 52.6 ± 12 | 44.7 ± 8.6 | 0.022 |
| ADP 5 late | 46.3 ± 15 | 36.8 ± 10.8 | 0.011 |

ADP: adenosine diphosphate;
AA: arachidonic acid;
ASA: aspirin;
COLL: collagen;
3: Compound 3;
p = value;
SD: standard deviation.

TABLE 2
Platelet aggregation in citrate-anticoagulated PRP (LTA) on
clopidogrel treatment with and without Compound 3.

| | CLOP (mean ± SD) | CLOP + 3 (mean ± SD) | P |
|---|---|---|---|
| AA max | 49.7 ± 28.2 | 1.2 ± 1.6 | 0.009 |
| AA late | 47.6 ± 28.2 | 0.5 ± 0.8 | 0.008 |
| COLL max | 72.6 ± 10.2 | 30.1 ± 18.1 | 0.005 |
| COLL late | 70.5 ± 10.5 | 28.7 ± 16.4 | 0.005 |
| U-46619 max | 71.6 ± 11.6 | 0.9 ± 0.9 | 0.005 |
| U-46619 late | 68.8 ± 13.8 | 0 ± 0 | 0.005 |
| ADP 20 max | 54.8 ± 13.3 | 44.3 ± 12.3 | 0.011 |
| ADP 20 late | 44.6 ± 18.9 | 33.3 ± 17.2 | 0.021 |
| ADP 5 max | 32.8 ± 17.2 | 22.8 ± 12.4 | 0.009 |
| ADP 5 late | 19.7 ± 21.9 | 9.7 ± 12.4 | 0.025 |

ADP: adenosine diphosphate;
AA: arachidonic acid;
CLOP: clopidogrel;
COLL: collagen;
3: Compound 3;
p = value;
SD: standard deviation.

TABLE 3
Platelet aggregation in citrate-anticoagulated PRP (LTA). Comparison
between aspirin and clopidogrel treatment with Compound 3.

| | ASA + 3 (mean ± SD) | CLOP + 3 (mean ± SD) | P |
|---|---|---|---|
| AA max | 2.3 ± 1.2 | 1.2 ± 1.6 | 0.056 |
| AA late | 1.7 ± 1.6 | 0.5 ± 0.8 | 0.052 |
| COLL max | 40.4 ± 15.5 | 30.1 ± 18.1 | 0.083 |
| COLL late | 39.1 ± 15.5 | 28.7 ± 16.4 | 0.059 |
| U-46619 max | 1.2 ± 0.7 | 0.9 ± 0.9 | 0.429 |
| U-46619 late | 0 ± 0 | 0 ± 0 | 1 |
| ADP 20 max | 67.6 ± 8.7 | 44.3 ± 12.3 | 0.005 |
| ADP 20 late | 59 ± 20.2 | 33.3 ± 17.2 | 0.011 |
| ADP 5 max | 44.7 ± 8.6 | 22.8 ± 12.4 | 0.008 |
| ADP 5 late | 36.8 ± 10.8 | 9.7 ± 12.4 | 0.005 |

ADP: adenosine diphosphate;
AA: arachidonic acid;
ASA: aspirin;
CLOP: clopidogrel;
COLL: collagen;
3: Compound 3;
p = value;
SD: standard deviation.

1. A method for reducing risk of cardiovascular event in a diabetic patient comprising administering to the patient a therapeutically effect amount of a pharmaceutically acceptable form of a compound which is (Z)-6-((2S,4S,5R)-2-(2-chlorophenyl)-4-(2-hydroxyphenyl)-1,3-dioxan-5-yl)hex-4-enoic acid in combination with administering to the patient a pharmaceutically effect amount of a pharmaceutically acceptable form of a P2Y12 inhibitor.

2. The method of claim 1, where the P2Y12 inhibitor is selected from cangrelor, prasugrel, ticagrelor, elinogrel, clopidogrel, or ticlopidine, and combinations thereof.

3. The method of claim 1, where (Z)-6-((2S,4S,5R)-2-(2-chlorophenyl)-4-(2-hydroxyphenyl)-1,3-dioxan-5-yl)hex-4-enoic acid and the P2Y12 inhibitor are administered simultaneously.

4. The method of claim 1, where (Z)-6-((2S,4S,5R)-2-(2-chlorophenyl)-4-(2-hydroxyphenyl)-1,3-dioxan-5-yl)hex-4-enoic acid and the P2Y12 inhibitor are administered sequentially.

5. The method of claim 1, where the therapeutically effective amount of (Z)-6-((2S,4S,5R)-2-(2-chlorophenyl)-4-(2-hydroxyphenyl)-1,3-dioxan-5-yl)hex-4-enoic acid is a dose from about 0.0001 mg to about 20 mg of (Z)-6-((2S,4S,5R)-2-(2-chlorophenyl)-4-(2-hydroxyphenyl)-1,3-dioxan-5-yl)hex-4-enoic acid per kg of the patient body weight per day.

6. The method of claim 1, where the therapeutically effective amount of (Z)-6-((2S,4S,5R)-2-(2-chlorophenyl)-4-(2-hydroxyphenyl)-1,3-dioxan-5-yl)hex-4-enoic acid is a dose of from about 0.001 mg to about 1 mg of (Z)-6-((2S,4S,5R)-2-(2-chlorophenyl)-4-(2-hydroxyphenyl)-1,3-dioxan-5-yl)hex-4-enoic acid per kg of the patient body weight per day.

7. The method of claim 1 where the cardiovascular event is myocardial infarction, thrombosis, a thrombotic disorder, stent triggered thrombus formation, stent induced restenosis, stent-triggered hyperplasia, pulmonary hypertension, atherosclerosis, diabetic nephropathy, retinopathy, diabetic retinopathy, peripheral arterial disease, lower limb circulation, pulmonary embolism, thrombus formation, hyperplasia, septic shock, preeclampsia, asthma, rhinitis, allergic rhinitis, tumor angiogenesis or metastasis.

8. A method for treating vascular inflammation in a diabetic patient comprising administering to the patient a therapeutically effect amount of a pharmaceutically acceptable form of a compound which is (Z)-6-((2S,4S,5R)-2-(2-chlorophenyl)-4-(2-hydroxyphenyl)-1,3-dioxan-5-yl)hex-4-enoic acid in combination with administering to the patient a pharmaceutically effect amount of a pharmaceutically acceptable form of a P2Y12 inhibitor.

9. A method for reducing vascular inflammation in a diabetic patient comprising administering to the patient a therapeutically effect amount of a pharmaceutically acceptable form of a compound which is (Z)-6-((2S,4S,5R)-2-(2-chlorophenyl)-4-(2-hydroxyphenyl)-1,3-dioxan-5-yl)hex-4-enoic acid in combination with administering to the patient a pharmaceutically effect amount of a pharmaceutically acceptable form of a P2Y12 inhibitor.

10. A method for reducing platelet reactivity in a diabetic patient comprising administering to the patient a therapeutically effect amount of a pharmaceutically acceptable form of a compound which is (Z)-6-((2S,4S,5R)-2-(2-chlorophenyl)-4-(2-hydroxyphenyl)-1,3-dioxan-5-yl)hex-4-enoic acid in combination with administering to the patient a pharmaceutically effect amount of a pharmaceutically acceptable form of a P2Y12 inhibitor.

11. A method for improving vascular function in a diabetic patient comprising administering to the patient a therapeutically effect amount of a pharmaceutically acceptable form of a compound which is (Z)-6-((2S,4S,5R)-2-(2-chlorophenyl)-4-(2-hydroxyphenyl)-1,3-dioxan-5-yl)hex-4-enoic acid in combination with administering to the patient a pharmaceutically effect amount of a pharmaceutically acceptable form of a P2Y12 inhibitor.

12. A method for reducing vascular oxidative stress in a diabetic patient comprising administering to the patient a therapeutically effect amount of a pharmaceutically acceptable form of a compound which is (Z)-6-((2S,4S,5R)-2-(2-chlorophenyl)-4-(2-hydroxyphenyl)-1,3-dioxan-5-yl)hex-4-enoic acid in combination with administering to the patient a pharmaceutically effect amount of a pharmaceutically acceptable form of a P2Y12 inhibitor.


SEQUENCE LISTING (16 sequences)

SEQ ID NO: 1 - DNA, 25 bp, Artificial Sequence
  Synthetic oligonucleotide
attagcctgaatgtgccataagact

SEQ ID NO: 2 - DNA, 23 bp, Artificial Sequence
  Synthetic oligonucleotide
acccacagtgcttgacacagaat

SEQ ID NO: 3 - DNA, 21 bp, Artificial Sequence
  Synthetic oligonucleotide
ggagcttcgtgtcctgtatgg

SEQ ID NO: 4 - DNA, 22 bp, Artificial Sequence
  Synthetic oligonucleotide
acattggagtctgctgggaatt

SEQ ID NO: 5 - DNA, 19 bp, Artificial Sequence
  Synthetic oligonucleotide
tggctgcaggacatgacaa

SEQ ID NO: 6 - DNA, 21 bp, Artificial Sequence
  Synthetic oligonucleotide
ccatgctacatttgccgaaga

SEQ ID NO: 7 - DNA, 25 bp, Artificial Sequence
  Synthetic oligonucleotide
ttgatactcccagtcttgtcattgc

SEQ ID NO: 8 - DNA, 23 bp, Artificial Sequence
  Synthetic oligonucleotide
tgactgtggagttttggctgttt

SEQ ID NO: 9 - DNA, 21 bp, Artificial Sequence
  Synthetic oligonucleotide
cattgtggccaaggagatctg

SEQ ID NO: 10 - DNA, 25 bp, Artificial Sequence
  Synthetic oligonucleotide
agtgagtgttcaagtcttcggagtt

SEQ ID NO: 11 - DNA, 21 bp, Artificial Sequence
  Synthetic oligonucleotide
caatggactccatcccactga

SEQ ID NO: 12 - DNA, 22 bp, Artificial Sequence
  Synthetic oligonucleotide
gatgaagagcttgtcccattcc

SEQ ID NO: 13 - DNA, 25 bp, Artificial Sequence
  Synthetic oligonucleotide
gcctacaagaaagtttgcctatctg

SEQ ID NO: 14 - DNA, 24 bp, Artificial Sequence
  Synthetic oligonucleotide
tgagctgtttcttcttccggtagt

SEQ ID NO: 15 - DNA, 27 bp, Artificial Sequence
  Synthetic oligonucleotide
ttggctcacaattaagaagtttaacac

SEQ ID NO: 16 - DNA, 25 bp, Artificial Sequence
  Synthetic oligonucleotide
gcaggtattattaaggaggatgcaa